SEQUENCE LISTING (19 sequences)

SEQ ID NO: 1 - DNA, 23 bp, Artificial
  hGAD65mut Forward primer
caccatggcatctccgggctctg

SEQ ID NO: 2 - DNA, 29 bp, Artificial
  hGAD65mut Reverse primer
ttattataaatcttgtccaaggcgttcta

SEQ ID NO: 3 - DNA, 23 bp, Artificial
  GAD67/65 Forward primer
caccatggcatcttccacgcctt

SEQ ID NO: 4 - DNA, 28 bp, Artificial
  GAD67/65 Reverse primer
tattataaatcttgtccaaggcgttcta

SEQ ID NO: 5 - DNA, 1761 bp, Artificial
  GAD65mut nucleotidic sequence
atggcatctccgggctctggcttttggtctttcgggtcggaagatggctctggggattccgagaatcccggcacagcgcgagcctggtgccaagtggctcagaagttcacgggcggcatcggaaacaaactgtgcgccctgctctacggagacgccgagaagccggcggagagcggcgggagccaacccccgcgggccgccgcccggaaggccgcctgcgcctgcgaccagaagccctgcagctgctccaaagtggatgtcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacaccgcatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttataataa

SEQ ID NO: 6 - DNA, 1762 bp, Artificial
  GAD67/65mut nucleotidic sequence
atggcatcttccacgccttcgcctgcaacctcctcgaacgcgggagcggatcctaatactaccaacctgcgtcctacaacatatgatacttggtgtggcgtagcccatggatgcaccagaaaactgggcctgaagatctgtggcttcttgcaaaggaccaatagcctggaagagaagagtcgtcttgtgagtgccttcagggagaggcaggcctccaagaacctgctttcctgtgaaaacagtgaccctggtgcccgcttcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacaccgcatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttattaataa

SEQ ID NO: 7 - DNA, 25 bp, Artificial
  GAD65 Forward primer
caccatggctagcccaggctccgga

SEQ ID NO: 8 - DNA, 29 bp, Artificial
  GAD65 Reverse primer
ttattataaatcttgtccaaggcgttcta

SEQ ID NO: 9 - DNA, 1795 bp, Artificial
  GAD65 nucleotidic sequence
atggctagcccaggctccggattttggtctttcgggtcggaagatggctctggggattccgagaatcccggcacagcgcgagcctggtgccaagtggctcagaagttcacgggcggcatcggaaacaaactgtgcgccctgctctacggagacgccgagaagccggcggagagcggcgggagccaacccccgcgggccgccgcccggaaggccgcctgcgcctgcgaccagaagccctgcagctgctccaaagtggatgtcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacaccgcatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttataataaccttgctcaccaagctgttccacttctctagaga

SEQ ID NO: 10 - DNA, 16 bp, Artificial
  M13 Forward primer
gtaaaacgacggccag

SEQ ID NO: 11 - DNA, 21 bp, Artificial
  1500R primer
caaacaccatctcatatcctt

SEQ ID NO: 12 - DNA, 17 bp, Artificial
  M13 Reverse primer
caggaaacagctatgac

SEQ ID NO: 13 - DNA, 18 bp, Artificial
  G690 Forward primer
tcattggctggccagggg

SEQ ID NO: 14 - DNA, 20 bp, Artificial
  Nuovo35S primer
aagatgcctctgccgacagt

SEQ ID NO: 15 - DNA, 18 bp, Artificial
  65int primer
cacacgccggcagcaggt

SEQ ID NO: 16 - DNA, 21 bp, Artificial
  GADh1 primer
gtttggagttggcagagtaat

SEQ ID NO: 17 - DNA, 19 bp, Artificial
  GADh2 primer
agacatttgtgtgctgagg

SEQ ID NO: 18 - DNA, 20 bp, Artificial
  ACT1 primer
atcccagttgctgacaatac

SEQ ID NO: 19 - DNA, 21 bp, Artificial
  ACT2 primer
ggcccgccatactggtgtgat

(19) United States
(12) Patent Application Publication
(10) Pub. No.: US 2011/0289630 A1
(43) Pub. Date: Nov. 24, 2011

(54) METHOD FOR THE EXPRESSION IN PLANT OF THE GLUTAMIC ACID DECARBOXYLASE (GAD65) AND RELATED EXPRESSION VECTORS

(75) Inventors: Linda Avesani, Verona (IT); Alberto Falorni, Perugia (IT); Mario Pezzotti, Gargagnago di S.Ambrogio (VR) (IT)

(73) Assignee: UNIVERSITA DEGLI STUDI DI VERONA, Verona (IT)

(21) Appl. No.: 13/003,857

(22) PCT Filed: Jul. 24, 2009
(86) PCT No.: PCT/IT2009/000330

§ 371 Date: Aug. 11, 2011

(30) Foreign Application Priority Data
Jul. 25, 2008 (IT) RM2008A000403

(51) Int. Cl. C12N 15/82; C12N 1/21; C12N 15/63

(52) U.S. Cl. 800/294; 435/320.1; 435/252.3; 435/252.33; 800/278

(57) ABSTRACT

The present invention concerns a method of expression in plant of glutamic acid decarboxylase (GAD65), particularly a mutated form of human GAD65 (GAD65mut), and expression vectors thereof.

The present invention concerns a method of expression in plant of glutamic acid decarboxylase (GAD65), particularly a mutated form of human GAD65 (GAD65mut) and expression vectors thereof.

Insulin-dependent Mellitus Diabetes or type 1 diabetes (T1DM) is a syndrome characterized by metabolic anomalies associated to the alteration of glucidic profile, often together with both acute and chronic complications. T1DM is characterized by very severe clinical picture and involves the administration of insulin just after the disease onset.

T1DM is an autoimmune disease resulting from the failure of the normal mechanisms responsible for the maintenance of the tolerance towards the autologous antigens, also named “self-tolerance”.

The insulin deficiency in diabetics results from the destruction of β cells which, in the pancreatic Langerhans' islands, are responsible of this hormone synthesis, consequently it is necessary a continuous substitution therapy involving the daily administration of insulin over all the life.

The destruction of β cells derives from an autoimmune type response resulting from the failure of the normal mechanisms responsible for the maintenance of the tolerance towards the autologous antigens. The disease symptoms generally appear when nearly all the β cells are now destroyed, whereby it is impossible to limit the damages to pancreatic tissue. This destruction process can take advantage of various mechanisms among which there are lysis mediated by T cells, B cells B, macrophages and auto-antibodies against the island cells.

In serum of suffering patients it is possible to find auto-antibodies against many island antigens. The analysis of these antibodies pointed out that the same recognize certain components of β cells, among which 65 kDa isoform of glutamic acid decarboxylase (GAD65) proved to be the most important (Hagopian et al., 1993). The auto-antibodies to GAD65 can be developed many years before the disease occurrence and this enzyme isoform is involved in other autoimmune diseases such as “stiff-man” syndrome and polyendocrine diseases.

In order to study insulin-dependent mellitus diabetes two animal models can be used: an “inbred” rat strain named BB (Bio-Breeding) and a diabetic not obese rat strain (NOD), developing a T lymphocyte mediated spontaneous insulite very similar to human one.

As a result from an immunological analysis in NOD rats it has been observed that the GAD65 is the highest primary auto-antigen, because T cells and antibodies against this molecule in the autoimmune response towards β cells are firstly developed and in more remarkable way than those against others components of same cells (Baekkeskov et al., 1982).

It has been observed that the autoimmune response is limited, at first, to a single GAD65 region, and secondary it is diffuse both intra-molecularly, towards other antigenic determinants in the same protein, and inter-molecularly, towards other antigens of β cells (ICA69, H carboxypeptidase and gangliosides). These auto-antigens are defined secondary because antibodies directed against these molecules are present in the serum of suffering patients in lower amount than those directed against primary auto-antigens such as GAD65, insulin and some tyrosine kynases (IA2, ICA512, IA2b).

The role of antibodies directed against GAD65 and other island antigens in T1DM pathogenesis however has not still been understood but their presence is an important marker for diagnosis and prediction of such disease.

The parenteral administration of human GAD65, i.e. the highest auto-antigen associated to insulin-dependent mellitus diabetes or type 1 diabetes (T1DM), can be used in order to induce immuno-tolerance and consequently prevent the disease development. Recent studies carried out by the Swedish Company Diamyd Medical demonstrated that two 20 μg parenteral administrations of purified human GAD65 in a four week period display meaningfully positive results in Phase II clinical trials carried out on pre-diabetic patients. Another possible approach for tolerance induction is the oral administration requiring the ingestion of remarkable amounts of protein antigen.

Currently, the process for the production of human GAD65 based on the use of “insect cell/baculovirus” expression system results in extremely high production costs of the molecule, i.e. 600.000-700.000 euro/g of GAD65.

It is apparent that a limiting factor for the clinical use of GAD65 aiming to tolerance induction is the production cost of the protein since in order to induce tolerance (above all orally) it is necessary that large amounts of auto-antigens, higher than those obtained from conventional fermentation or protein synthesis systems, to be available. Further, the expression in bacterial systems unable to carry out appropriate refolding of the protein and correct glycosylation, leads to the formation of inclusion bodies and a remarkable reduction in the recovery levels of usable protein antigens.

Studies aiming to the production and characterization of transgenic tobacco and carrot plants expressing human GAD65 have been carried out (Porceddu et al., 1999). Using the “immunogold labelling” and electron microscopy techniques on transgenic tobacco tissues it has been observed that in planta expressed hGAD65 is mostly associated to mitochondrial and chloroplast membranes. Moreover, a radioimmuno-assay analysis (RIA) has evidenced that in planta expressed hGAD65 is immunoreactive, i.e. it is recognized by auto-antibodies occurring in the sera of T1DM suffering patients and maintains its enzymatic activity. As to the expression levels of obtained hGAD65 the same are from 0.01% to 0.04% of soluble total proteins (PST).

A further study (Avesani et al., 2003) has predicted the use of an expression vector based on Agrobacterium (stable transformation) in order to increase the hGAD65 expression by means of retention in cytosolic environment. Therefore, GAD critical residues, responsible of the anchorage of the protein to membranes, localized in the enzyme N-terminal region, have been modified. A chimerical molecule for the production of transgenic plants expressing a GAD cytosolic form, has been constructed by replacement of GAD65 N-terminal region with corresponding region from Rattus norvegicus GAD67. GAD 67 kDa isoform is not a T1DM auto-antigen and does not have in the N-terminal region the residues responsible for GAD65 sub-cellular localization whereby it is localized in the cytosol of rat cells. It has been therefore produced a GAD67/65 chimerical cDNA wherein the first GAD65 87 amino acids have been replaced with the corresponding GAD67 amino acids. GAD67/65 chimerical molecule expressed in tobacco remains immunoreactive, as expected, because the epitopes recognized by antibodies from T1DM affected patients are localized in the central and carboxy-terminal region of the protein.

“Immunogold labelling” and electron microscopy techniques on leaf tissues have confirmed that GAD67/65 occurs mostly at cytosolic level. In leaves of transgenic plants the immunoreactive protein amount on average is between 0.05% and 0.03% of the PST. This demonstrates that GAD67/65 stably transformed plants have highest expression levels which are fivefold higher than maximum obtained with human GAD65 (0.04% of the PST). By comparing produced protein amount and accumulation of corresponding transcript it seems that human GAD is much more stable in the cytosol environment than in membrane association.

In the same study moreover a system for the transient expression of hGAD65 based on a viral modified vector has been used: i.e. Potato Virus X (PVX). Using RIA assays hGAD65 average levels equals to 2.16% of PST occurring in leaves from N. benthamiana infected plants have been measured. Such levels of recombinant protein expression are ten times higher than the maximum expression levels obtained from a stable transformation (0.19% of PST). The quantification of hGAD65 content in benthamiana N. plants has been carried out only for the first plants infected with the viral transcript, while for successive infections such expression levels are drastically reduced. This strategy has been proved to be very promising when the obtained expression levels of recombinant protein are considered, but it is not applicable on large scale due to the difficult virus control.

From above it is apparent the need to provide a method allowing that, in a cheap and scalable way, overcoming disadvantages resulting from prior art approaches, large amounts of GAD65 immunoreactive protein to be produced.

In order to increase the GAD65 expression levels the authors of the present invention provided an approach for modification of protein cellular localization using constructs expressing a mutated form of hGAD65 (hGAD65mut). This mutated form is obtained by replacing, using site-specific mutagenesis, a codon triplet encoding for Lys396 amino acid with a codon triplet encoding for Arg amino acid. Replaced Lys is localized in the GAD catalytic site and it is the amino acid responsible for pyridoxal-phosphate (PLP) binding, indispensable co-factor for the enzyme activity.

The mutated form of the enzyme (hGAD65mut) already had been described in a previous study concerning the in vitro (therefore not in vivo) protein expression using a transcription/translation system from the molecule cDNA wherein it has been demonstrated that with such mutation the molecule immunoreactivity is unaltered and the in vitro enzymatic activity of the protein is eliminated (Hampe et al., 2001).

Particularly, the authors of the invention produced three constructs wherein the hGAD65 mutated form (hGAD65mut) has been engineered in order to drive the protein in different sub-cellular compartments of the plant cell. At our knowledge currently the mutated form of hGAD65 never has been expressed in plant.

The three constructs obtained from the enzyme mutated form are the following ones:

hGAD65mut, for the protein anchoring to chloroplast and mitochondrial membranes, as obtained by the previous transformation with hGAD65;

GAD67/65mut, for the retaining of the protein in the cytosol environment, as obtained by the previous transformation with GAD67/65;

hGAD65, for the anchoring of the molecule enzymatically active form to chloroplast and mitochondrial membranes.

Among these constructs the authors of the present invention have demonstrated that hGAD65mut and GAD67/65mut expressing constructs unexpectedly allow maximum levels of GAD65 expression in plant to be obtained. Further it would seem that the transformation using these two hGAD65mut and GAD67/65mut expressing constructs is particularly advantageous because it would result in a greater stability of the protein rather than increased transcript levels. Thus expressed protein, also in the chimerical form with GAD67/65mut, maintains its immunoreactive properties as demonstrated successively and it can be used as an antigen for the screening of auto-antibodies or as a therapeutic protein in diabetic subjects.

It is therefore an object of the present invention a method for the production in plant of a mutated form of the glutamic acid decarboxylase auto-antigen (GAD65mut; SEQ ID NO:5)) or a chimera thereof comprising the following steps:

a) transformation of a plant or a portion or tissue thereof, with an expression vector comprising the sequence of the mutated form of human GAD65, characterised by the substitution of Lys aminoacid at position 396 with Arg aminoacid, under the control of a plant constitutive promoter and terminator using Agrobacterium tumefaciens competent cells;
b) plant growth and auto-antigen expression;
c) auto-antigen expressing plant tissue harvesting.

According to a preferred embodiment the method of the invention further comprises a purification step by column chromatography of the mutated form of glutamic acid decarboxylase auto-antigen (GAD65mut; SEQ ID NO: 5) from plant tissue collected in step c).

According to an alternative embodiment of the method of the invention, said chimera is GAD67/65mut having SEQ ID NO: 6. This chimerical molecule expresses a GAD cytosol form through the substitution of the N-terminal region of human GAD65mut (having Lys 396 amino acid replaced with Arg amino acid) with the corresponding region of Rattus norvegicus GAD67. It has been therefore provided a chimerical GAD67/65mut cDNA wherein the first 87 amino acids of the human GAD65mut have been replaced with the corresponding amino acids of rat GAD67.

According to a preferred embodiment the plant is a tobacco plant, more preferably is Nicotiana tabacum.

According to a further preferred embodiment of the inventive method, said plant tissue is a leaf.

According to a preferred embodiment of the inventive method, said promoter is CaMV 35S (P35S) and said terminator is CaMV 35S terminator (T35S).

The use of a plant or plant tissue transformed with the expression vector comprising the sequence of the mutated form of human GAD65 or a chimera thereof, characterized by the substitution of Lys amino acid at position 396 with Arg amino acid under the control of a plant constitutive promoter, as a bioreactor for the production of GAD65 auto-antigen, represents a further object of the present invention.

According to an alternative embodiment, said chimera is GAD67/65mut.

According to a preferred embodiment the plant is a tobacco plant, more preferably Nicotiana tabacum.

According to a further preferred embodiment of the inventive method, said plant tissue is a leaf.

Further the invention refers to an expression vector comprising the sequence of the mutated form of human GAD65 or a chimera thereof, characterized by the substitution of Lys amino acid at position 396 with Arg amino acid under the control of a plant constitutive promoter and terminator. According to an alternative embodiment of said expression vector, said chimera is GAD67/65mut.

According to a particularly preferred embodiment of the expression vector of the invention, said constitutive promoter is CaMV 35S and said terminator is CaMV 35S terminator

Further the invention refers to prokaryotic or eukaryotic competent cells transformed with the expression vector as above defined. Said prokariotic cells are preferably from Agrobacterium tumefaciens or Escherichia coli strains.

Finally, it is an object of the present invention the use of above mentioned expression vector or competent cells for the transformation of a plant or plant tissue. According to a preferred embodiment the plant is a tobacco plant, more preferably Nicotiana tabacum.

According to a further preferred embodiment of the inventive method, said plant tissue is a leaf.

In the following experimental section the GAD65mut and GAD67/65mut chimera nucleotide sequences, preferably used in the context of the present invention, are illustrated and to be intended in an exemplary but not limitative way.

The present invention now will be described by an illustrative, but not limitative way, according to preferred embodiments, particularly with reference to included drawings wherein:

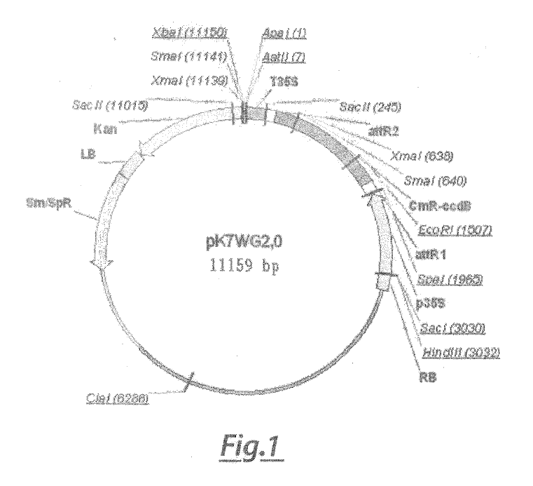

FIG. 1 shows a schematic drawing of the expression vector pK7WG2;

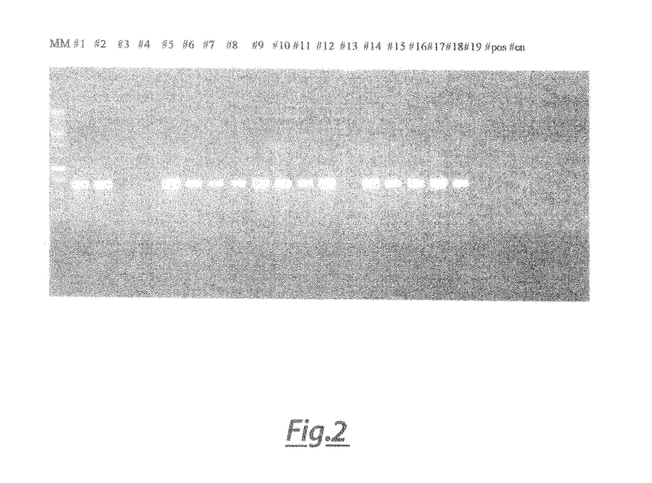

FIG. 2 shows an example of electrophoresis run for DNA from pK7WG2.G65mut vector transformed plants; MM: molecular marker; “+”: positive control; “−”: negative control;

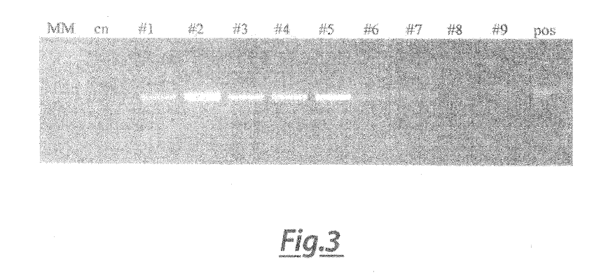

FIG. 3 shows an example of electrophoresis run for DNA from pK7WG2.G65mut vector transformed plants; MM: molecular marker; “+”: positive control; “−”: negative control;

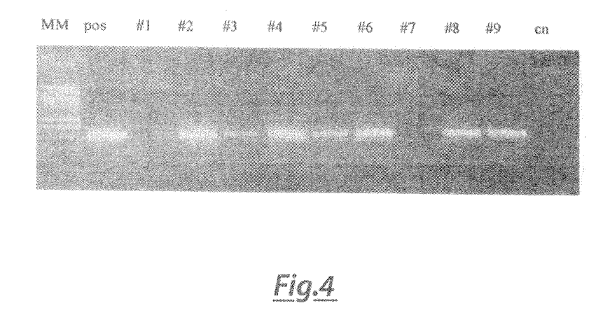

FIG. 4 shows an example of electrophoresis run for DNA from pK7WG2.GAD65 vector transformed plants; MM: molecular marker; “+”: positive control; “−”: negative control;

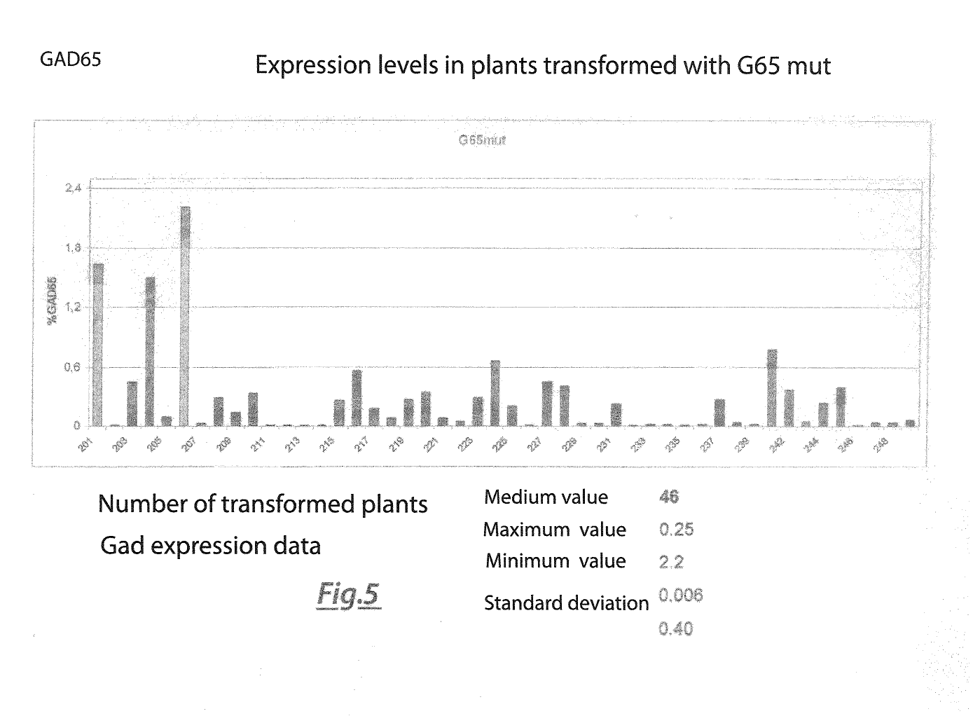

FIG. 5 shows the GAD65 expression levels in pK7WG2.G67/65mut construct transformed plants;

FIG. 5 shows the GAD65 expression levels in pK7WG2.G65 construct transformed plants;

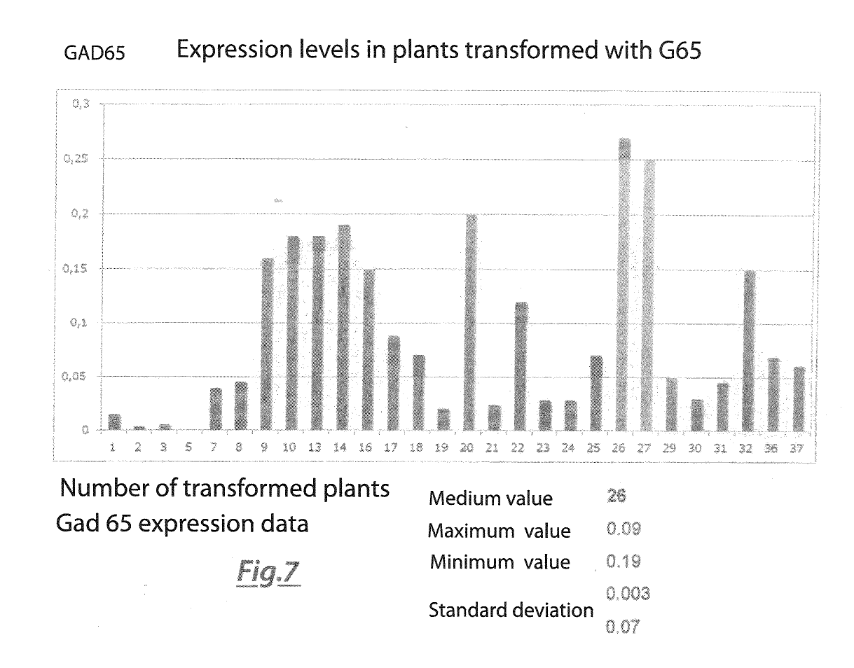

FIG. 7 shows a boxplot diagram of GAD65mut different expression levels as evaluated by RIA, and obtained with the various constructs used for the transformation; the components of the boxplot diagram are the following ones: horizontal, median line; rectangle: interval between first and third quartile; moustaches, correspond to values distant 1.5 fold the inter-quartile distance from the first and third quartile, respectively; in the drawing also values falling outside of the interval delimited by two lines (moustaches) as isolated points are present;

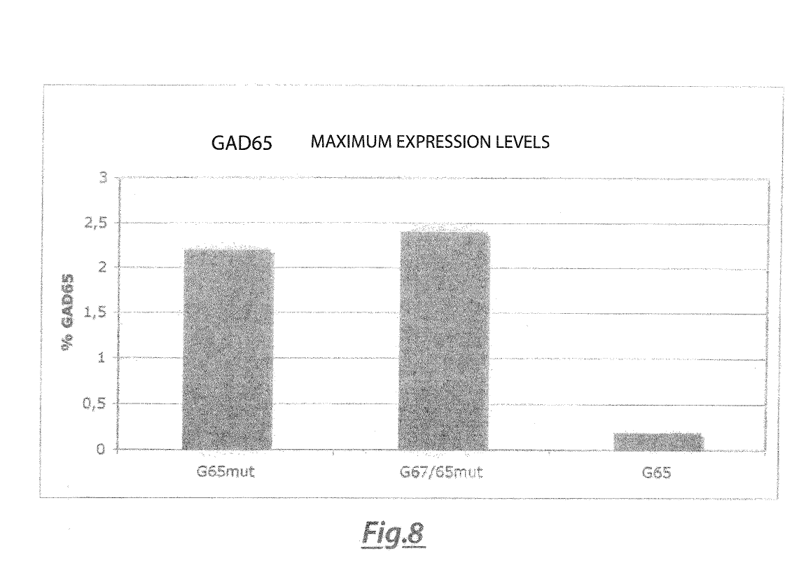

FIG. 8 shows a comparison of GAD65mut expression levels as obtained for each construct used;

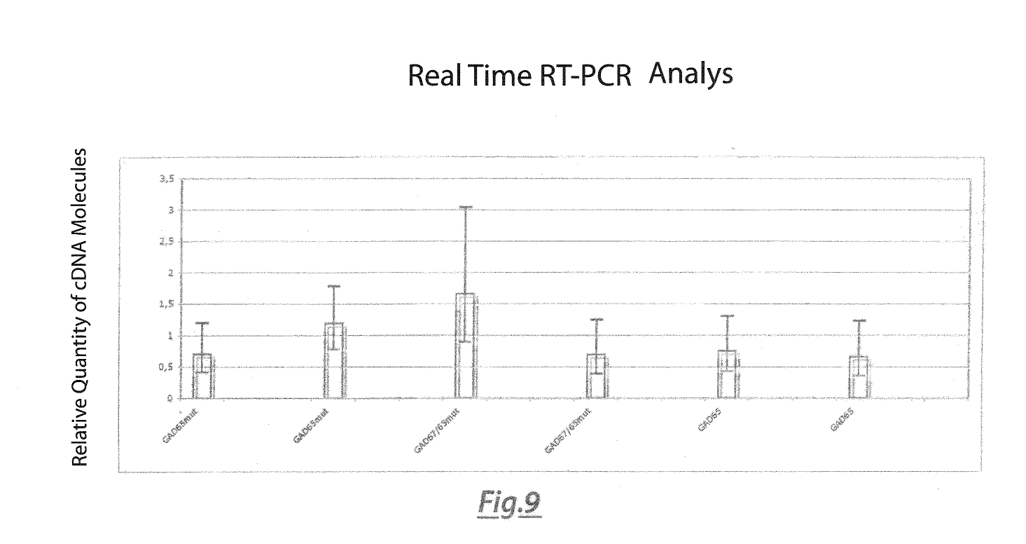

FIG. 9 shows a comparison of expression levels for GAD65 (Porceddu et al., 1999), GAD67/65 (Avesani et al., 2003), GAD65mut and GAD67/65mut;

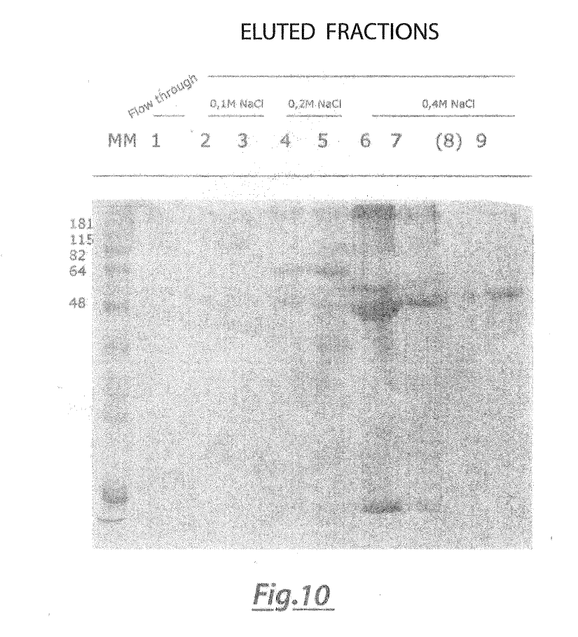

FIG. 10 shows the quantification of GAD65mut transcript (RT-PCR) relating to the actin in GAD65mut, GAD67/65mut and GAD65 transformed plants expressing the highest levels of recombinant GAD65mut;

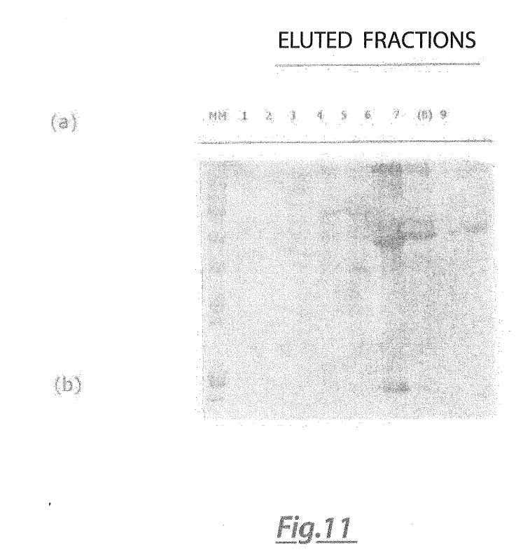

FIG. 11 shows Coomassie Blue staining (a) and Western blot analysis (b) with primary, hGAD65 C-terminal region specific, GC3108 (Affiniti) antibody; MM: molecular marker; 1,2,3: fractions collected as a result of the elution with NaCl 0.1 M added buffer; 4,5: fractions collected as a result of the elution with NaCl 0.2 M added buffer; 6,7,9: fractions collected as a result of the elution with NaCl 0.4 M added buffer.

### EXAMPLE 1

### Transformation of Tobacco Plants Using Constructs Comprising Mutated and not Mutated GAD65

Below materials and methods used for the construction of the vectors for the expression of these molecules and techniques used for the transformation of tobacco plants in order to express said molecules are reported.

### Materials and Methods

### Construction of the Expression Vectors

The three constructs obtained from enzyme mutated form are the following:

hGAD65mut, for the protein anchoring to chloroplast and mitochondrial membranes, as obtained by the previous transformation with hGAD65;

GAD67/65mut, for the retaining of the protein in the cytosol environment, as obtained by the previous transformation with GAD67/65;

GAD65, for the same previously obtained sub-cellular localization to be used as comparison in this expression system.

In the following is reported the experimental procedure used in order to construct the vector for the in planta expression, consisting essentially of:

1) engineering of the interest molecule by means of PCR reactions;

2) cloning of constructed molecules in pENTR™/D-TOPO® (Invitrogen) vector;

3) LR recombination of vectors containing the interest molecules cloned in pENTR™/D-TOPO® and in planta expression vector pK7WG2, respectively.

### Engineering of the Interest Molecules by Means of PCR Reactions

### GAD65mut and GAD67/65mut

GAD65mut and GAD67/65mut were available in our laboratory in pBluescript vector.

For the amplification of these molecules specific primers having at 5′ end of forward primer a clamp of four nucleotides (CACC) indispensable for the pairing to overhang sequence (GTGG) occurring in pENTR™TOPO® vector have been used.

For hGAD65mut construct the following primers have been used:

(SEQ ID NO: 1)
Gad65for: CACCATGGCATCTCCGGGCTCTG
and
(SEQ ID NO: 2)
Gadrev: TTATTATAAATCTTGTCCAAGGCGTTCTA

while for GAD67/65 construct the following primers have been used

(SEQ ID NO: 3)
G67/65for: CACCATGGCAtcttccacgcctt
and
(SEQ ID NO: 4)
Gadrev: TATTATAAATCTTGTCCAAGGCGTTCTA.

For all the amplifications a Taq polymerase with “proof-reading” activity has been used.

Below the nucleotide sequences of used molecules are reported.

GAD65mut
(SEQ ID NO: 5)
ATGGCATCTCCGGGCTCTGGCTTTTGGTCTTTCGGGTCGGAAGATGGCT
CTGGGGATTCCGAGAATCCCGGCACAGCGCGAGGGCCTGGTGCCAAGTG
GCTCAGAAGTTCACGCGGCATCGGAAACAAACTGTGCGCCCTGCTCTAC
GGAGACGCCGAGAAGCCGGCGGAGAGCGGCGGGAGCCAACCCCCGCGGG
CCGCCGCCCGGAAGGCCGCCTGCGCCTGCGACCAGAAGCCCTGCAGCTG
CTCCAAAGTGGATGTCAACTACGCGTTTCTCCATGCAACAGACCTGCTG
CCGGCGTGTGATGGAGAAAGGCCCACTTTGGCGTTTCTGCAAGATGTTA
TGAACATTTTACTTCAGTATGTGGTGAAAAGTTTCGATAGATCAACCAA
AGTGATTGATTTCCATTATCCTAATGAGCTTCTCCAAGAATATAATTGG
GAATTGGCAGACCAACCACAAAATTTGGAGGAAATTTTGATGCATTGCC
AAACAACTCTAAAATATGCAATTAAAACAGGGCATCCTAGATACTTCAA
TCAACTTTCTACTGGTTTGGATATGGTTGGATTAGCAGCAGACTGGCTG
ACATCAACAGCAAATACTAACATGTTCACCTATGAAATTGCTCCAGTAT
TTGTGCTTTTGGAATATGTCACACTAAAGAAAATGAGAGAAATCATTGG
CTGGCCAGGGGGCTCTGGCGATGGGATATTTTCTCCCGGTGGCGCCATA
TCTAACATGTATGCCATGATGATCGCACGCTTTAAGATGTTCCCAGAAG
TCAAGGAGAAAGGAATGGCTGCTCTTCCCAGGCTCATTGCCTTCACGTC
TGAACATAGTCATTTTTCTCTCAAGAAGGGAGCTGCAGCCTTAGGGATT
GGAACAGACAGCGTGATTCTGATTAAATGTGATGAGAGAGGGAAAATGA
TTCCATCTGATCTTGAAAGAAGGATTCTTGAAGCCAAACAGAAAGGGTT
TGTTCCTTTCCTCGTGAGTGCCACAGCTGGAACCACCGTGTACGGAGCA
TTTGACCCCCTCTTAGCTGTCGCTGACATTTGCAAAAAGTATAAGATCT
GGATGCATGTGGATGCAGCTTGGGGTGGGGGATTACTGATGTCCCGAAA
ACACAAGTGGAAACTGAGTGGCGTGGAGAGGGCCAACTCTGTGACGTGG
AATCCACACCGCATGATGGGAGTCCCTTTGCAGTGCTCTGCTCTCCTGG
TTAGAGAAGAGGGATTGATGCAGAATTGCAACCAAATGCATGCCTCCTA
CCTCTTTCAGCAAGATAAACATTATGACCTGTCCTATGACACTGGAGAC
AAGGCCTTACAGTGCGGACGCCACGTTGATGTTTTTAAACTATGGCTGA
TGTGGAGGGCAAAGGGGACTACCGGGTTTGAAGCGCATGTTGATAAATG
TTTGGAGTTGGCAGAGTATTTATACAACATCATAAAAAACCGAGAAGGA
TATGAGATGGTGTTTGATGGGAAGCCTCAGCACACAAATGTCTGCTTCT
GGTACATTCCTCCAAGCTTGCGTACTCTGGAAGACAATGAAGAGAGAAT
GAGTCGCCTCTCGAAGGTGGCTCCAGTGATTAAAGCCAGAATGATGGAG
TATGGAACCACAATGGTCAGCTACCAACCCTTGGGAGACAAGGTCAATT
TCTTCCGCATGGTCATCTCAAACCCAGCGGCAACTCACCAAGACATTGA
CTTCCTGATTGAAGAAATAGAACGCCTTGGACAAGATTTATAATAA
GAD67/65mut
(SEQ ID NO: 6)
ATGGCATCTTCCACGCCTTCGCCTGCAACCTCCTCGAACGCGGGAGCGG
ATCCTAATACTACCAACCTGCGTCCTACAACATATGATACTTGGTGTGG
CGTAGCCCATGGATGCACCAGAAAACTGGGCCTGAAGATCTGTGGCTTC
TTGCAAAGGACCAATAGCCTGGAAGAGAAGAGTCGTCTTGTGAGTGCCT
TCAGGGAGAGGCAGGCCTCCAAGAACCTGCTTTCCTGTGAAAACAGTGA
CCCTGGTGCCCGCTTCAACTACGCGTTTCTCCATGCAACAGACCTGCTG
CCGGCGTGTGATGGAGAAAGGCCCACTTTGGCGTTTCTGCAAGATGTTA
TGAACATTTTACTTCAGTATGTGGTGAAAAGTTTCGATAGATCAACCAA
AGTGATTGATTTCCATTATCCTAATGAGCTTCTCCAAGAATATAATTGG
GAATTGGCAGACCAACCACAAAATTTGGAGGAAATTTTGATGCATTGCC
AAACAACTCTAAAATATGCAATTAAAACAGGGCATCCTAGATACTTCAA
TCAACTTTCTACTGGTTTGGATATGGTTGGATTAGCAGCAGACTGGCTG
ACATCAACAGCAAATACTAACATGTTCACCTATGAAATTGCTCCAGTAT
TTGTGCTTTTGGAATATGTCACACTAAAGAAAATGAGAGAAATCATTGG
CTGGCCAGGGGGCTCTGGCGATGGGATATTTTCTCCCGGTGGCGCCATA
TCTAACATGTATGCCATGATGATCGCACGCTTTAAGATGTTCCCAGAAG
TCAAGGAGAAAGGAATGGCTGCTCTTCCCAGGCTCATTGCCTTCACGTC
TGAACATAGTCATTTTTCTCTCAAGAAGGGAGCTGCAGCCTTAGGGATT
GGAACAGACAGCGTGATTCTGATTAAATGTGATGAGAGAGGGAAAATGA
TTCCATCTGATCTTGAAAGAAGGATTCTTGAAGCCAAACAGAAAGGGTT
TGTTCCTTTCCTCGTGAGTGCCACAGCTGGAACCACCGTGTACGGAGCA
TTTGACCCCCTCTTAGCTGTCGCTGACATTTGCAAAAAGTATAAGATCT
GGATGCATGTGGATGCAGCTTGGGGTGGGGGATTACTGATGTCCCGAAA
ACACAAGTGGAAACTGAGTGGCGTGGAGAGGGCCAACTCTGTGACGTGG
AATCCACACCGCATGATGGGAGTCCCTTTGCAGTGCTCTGCTCTCCTGG
TTAGAGAAGAGGGATTGATGCAGAATTGCAACCAAATGCATGCCTCCTA
CCTCTTTCAGCAAGATAAACATTATGACCTGTCCTATGACACTGGAGAC
AAGGCCTTACAGTGCGGACGCCACGTTGATGTTTTTAAACTATGGCTGA
TGTGGAGGGCAAAGGGGACTACCGGGTTTGAAGCGCATGTTGATAAATG
TTTGGAGTTGGCAGAGTATTTATACAACATCATAAAAAACCGAGAAGGA
TATGAGATGGTGTTTGATGGGAAGCCTCAGCACACAAATGTCTGCTTCT
GGTACATTCCTCCAAGCTTGCGTACTCTGGAAGACAATGAAGAGAGAAT
GAGTCGCCTCTCGAAGGTGGCTCCAGTGATTAAAGCCAGAATGATGGAG
TATGGAACCACAATGGTCAGCTACCAACCCTTGGGAGACAAGGTCAATT
TCTTCCGCATGGTCATCTCAAACCCAGCGGCAACTCACCAAGACATTGA
CTTCCTGATTGAAGAAATAGAACGCCTTGGACAAGATTTATTAATAA

### GAD65

GAD65 was available in our laboratory in pBluescript vector.

For the amplification of this molecule specific primers having at 5′ end of forward primer a clamp of four nucleotides (CACC) indispensable for the pairing to overhang sequence (GTGG) occurring in pENTR™TOPO® vector have been used.

For the generation of the construct the following primers have been used:

(SEQ ID NO: 7)
Gad65for: CACCATGGCTAGCCCAGGCTCCGGAe
(SEQ ID NO: 8)
Gadrev: TTATTATAAATCTTGTCCAAGGCGTTCTA

For all the amplifications a Taq polymerase with “proof-reading” activity has been used.

Below the nucleotide sequence of used molecule is reported.

GAD65:
(SEQ ID NO: 9)
ATGGCTAGCCCAGGCTCCGGATTTTGGTCTTTCGGGTCGGAAGATGGCT
CTGGGGATTCCGAGAATCCCGGCACAGCGCGAGCCTGGTGCCAAGTGGC
TCAGAAGTTCACGGGCGGCATCGGAAACAAACTGTGCGCCCTGCTCTAC
GGAGACGCCGAGAAGCCGGCGGAGAGCGGCGGGAGCCAACCCCCGCGGG
CCGCCGCCCGGAAGGCCGCCTGCGCCTGCGACCAGAAGCCCTGCAGCTG
CTCCAAAGTGGATGTCAACTACGCGTTTCTCCATGCAACAGACCTGCTG
CCGGCGTGTGATGGAGAAAGGCCCACTTTGGCGTTTCTGCAAGATGTTA
TGAACATTTTACTTCAGTATGTGGTGAAAAGTTTCGATAGATCAACCAA
AGTGATTGATTTCCATTATCCTAATGAGCTTCTCCAAGAATATAATTGG
GAATTGGCAGACCAACCACAAAATTTGGAGGAAATTTTGATGCATTGCC
AAACAACTCTAAAATATGCAATTAAAACAGGGCATCCTAGATACTTCAA
TCAACTTTCTACTGGTTTGGATATGGTTGGATTAGCAGCACTGACATCA
ACAGCAAATACGACTGGTAACATGTTCACCTATGAAATTGCTCCAGTAT
TTGTGCTTTTGGAATATGTCACACTAAAGAAAATGAGAGAAATCATTGG
CTGGCCAGGGGGCTCTGGCGATGGGATATTTTCTCCCGGTGGCGCCATA
TCTAACATGTATGCCATGATGATCGCACGCTTTAAGATGTTCCCAGAAG
TCAAGGAGAAAGGAATGGCTGCTCTTCCCAGGCTCATTGCCTTCACGTC
TGAACATAGTCATTTTTCTCTCAAGAAGGGAGCTGCAGCCTTAGGGATT
GGAACAGACAGCGTTTCTGATTAAATGTGATGAGAGAGGGAAAATGATT
CCATCGATGATCTTGAAAGAAGGATTCTTGAAGCCAAACAGAAAGGGTT
TGTTCCTTTCCTCGTGAGTGCCACAGCTGGAACCACCGTGTACGGAGCA
TTTGACCCCCTCTTAGCTGTCGCTGACATTTGCAAAAAGTATAAGATCT
GGATGCATGTGGATGCAGCTTGGGGTGGGGGATTACTGATGTCCCGAAA
ACACAAGTGGAAACTGAGTGGCGTGGAGAGGGCCAACTCTGTGACGTGG
AATCCACACCGCATGATGGGAGTCCCTTTGCAGTGCTCTGCTCTCCTGG
TTAGAGAAGAGGGATTGATGCAGAATTGCAACCAAATGCATGCCTCCTA
CCTCTTTCAGCAAGATAAACATTATGACCTGTCCTATGACACTGGAGAC
AAGGCCTTACAGTGCGGACGCCACGTTGATGTTTTTAAACTATGGCTGA
TGTGGAGGGCAAAGGGGACTACCGGGTTTGAAGCGCATGTTGATAAATG
TTTGGAGTTGGCAGAGTATTTATACAACATCATAAAAAACCGAGAAGGA
TATGAGATGGTGTTTGATGGGAAGCCTCAGCACACAAATGTCTGCTTCT
GGTACATTCCTCCAAGCTTGCGTACTCTGGAAGACAATGAAGAGAGAAT
GAGTCGCCTCTCGAAGGTGGCTCCAGTGATTAAAGCCAGAATGATGGAG
TATGGAACCACAATGGTCAGCTACCAACCCTTGGGAGACAAGGTCAATT
TCTTCCGCATGGTCATCTCAAACCCAGCGGCAACTCACCAAGACATTGA
CTTCCTGATTGAAGAAATAGAACGCCTTGGACAAGATTTATAATAACCT
TGCTCACCAAGCTGTTCCACTTCTCTAGAGA

Cloning in pENTER™ TOPO® Vector

After the obtainment of three amplicons as above described, a topoisomerase based reaction using pENTER™ TOPO® cloning vector has been carried out. By using the products from said reaction it has been possible to carry out the transformation of E. coli competent cells by “thermal shock”. The cells grown in kanamycin containing selective medium have been later analyzed by colony PCR using the primers:

| | |
|---|---|
| M13for: GTAAAACGACGGCCAG | (SEQ ID NO: 10) |
| and | |
| 1500R: CAAACACCATCTCATATCCTT, | (SEQ ID NO: 11) |
| and | |
| M13rev: CAGGAAACAGCTATGAC | (SEQ ID NO: 12) |
| and | |
| G690F: TCATTGGCTGGCCAGGGG. | (SEQ ID NO: 13) |

After electrophoresis on agarose gel, the height of the gel visualized PCR related band with M13 for and 1500R primers corresponds to expected one depending on analyzed construct.

After detection of PCR positive colonies inocula are carried out and DNA by means of minipreparations from insert containing cells is extracted. Plasmids extracted from PCR positive colonies corresponding to individual constructs have been sequenced and all display the correct sequence.

### Construction of Vectors for the in Planta Expression

Below reported vectors have been obtained from pK7WG2 expression vector, realized at Division of Functional Genomics of “Plant Systems Biology” Department at the University of Ghent. pK7WG2 used expression vector is schematically reported in FIG. 1.

The used vector has the following structural DNA components for the expression regulation of the interest gene:

Cauliflower Mosaic Virus (CaMV) promoter 35S;

Cauliflower mosaic virus (CaMV) terminator 35S.

Further the vector has the marker gene for kanamycin neomycin phosphotransferase II (nptII) resistance under the control of NOS promoter and terminator of A. tumefaciens nopaline synthase gene.

After the obtainment of previously described pEnter vectors it has been possible to perform LR recombination reaction using GATEWAY® (Invitrogen) cloning technology. Using pK7WG2 as destination vector pEnter.GAD65, pEnter.GAD65mut and pEnter.GAD67/65mut vectors have been used separately.

After the recombination reactions have been completed E. coli competent cells have been transformed.

Plasmidic DNA, extracted from colonies grown in streptomycin containing selective medium, has been controlled by a PCR reaction with New35S: AAGATGCCTCTGCCGACAGT (SEQ ID NO: 14) and 65int: CACACGCCGGCAGCAGGT (SEQ ID NO: 15) primer pair. PCR positive colonies have been used successively for the transformation of A. tumefaciens.

### Transformation of Tobacco Plants

The three vectors (pK7WG2.GAD65, pK7WG2.G65mut and pK7WG2.G67/65mut) obtained after purification using minipreparations, are employed in order to transform, by electroporation, A. tumefaciens EHA105 competent cells.

Vector containing colonies have been used successively for the tobacco plant transformation.

Leaves from Nicotiana tabacum plants, Petit Havana SR1 variety, have been transformed with leaf disc infection method using A. tumefaciens colonies containing described vectors.

Four infection cycles for the above listed vectors have been performed. An infection cycle with pBIN without insert has been used as negative control.

Approximately two months after the leaf disc infection with A. tumefaciens the seedlings, in vitro regenerated and selected by growing in kanamycin containing MS medium, have been transferred into greenhouse under controlled conditions.

### Results

Ex vitro regenerated plants were as below:

55 N. tabacum plants infected with A. tumefaciens containing the transformed pK7WG2.5PGAD67/65mutKDEL vector;

68 N. tabacum plants infected with A. tumefaciens containing the vector pK7WG2.G65mut;

63 N. tabacum plants infected with A. tumefaciens containing the vector pK7WG2.G67/65mut;

37 N. tabacum plants infected with A. tumefaciens containing the vector pK7WG2.G65.

5 N. tabacum plants infected with A. tumefaciens containing the pBin19 without insert.

### Molecular Characterization of Regenerated Plants

### PCR

Firstly genomic DNA has been extracted from leaf tissue of all ex vitro regenerated plants.

In order to verify that the plants had the T-DNA integrated in the genome a PCR analysis has been carried out using DNA extracted from each plant and the primer combination new35S and 65int (New35S: AAGATGCCTCTGCCGACAGT (SEQ ID NO: 14); 65int: CACACGCCGGCAGCAGG) (SEQ ID NO: 15).

In Table 1 the total of PCR positive plants is reported. In FIGS. 2-4 electrophoresis run examples of PCR products from plants transformed with the vectors used for N tabacum transformation are reported.

TABLE 1
PCR positive plants

| | TOTAL PCR | % PCR POSITIVE |
|---|---|---|
| CONSTRUCT | POSITIVE PLANTS | PLANTS |
| pK7WG2.G65 | 34 | 67.9% |
| pK7WG2.G65mut | 56 | 82.3% |
| pK7WG2.G67/65mut | 50 | 79.3% |

### EXAMPLE 2

### Analysis of GAD65mut Expression in Planta

### Materials and Methods

### Protein Extraction and RIA

For the protein extraction the leaf tissue from each sample has been ground with pestle in liquid nitrogen and the obtained powder homogenised in John extraction buffer (40 mM Hepes pH 7, 3, 5 mM EDTA, CHAPS 1.5%, 5 mM DTT).

Total protein amount in each protein extract has been quantified by means of Bradford colorimetric method.

Protein extracts form each transformed plant have been analysed by fluid phase “radioimmunoassay” (RIA) at Dipartimento di Medicina Interna e Scienze Endocrine e Metaboliche di Perugia, in order to assess whether modified GAD according to all previously described ways retained immunoreactivity, i.e. conformational regions serum antibodies from insulin dependent diabetes patients are directed to, and, in such case, to know the amount produced from transformed plants.

Two “radioimmunoassay” analyses for GAD quantification have been carried out: in the first all the transformed plants have been examined, while in the second, in order to reconfirm obtained data, have been analysed the plants for which in previous analysis higher values for recombinant protein expression had been obtained.

### RT-PCR

For each construct used for the transformation, two plants expressing the highest levels of recombinant protein, evaluated by RIA analysis, have been selected and analyzed by real Time RT-PCR in order to confront the accumulation levels of transgene transcript. This analysis has been carried out aiming to test whether the different levels of recombinant protein accumulation resulted from different levels of transgene transcript accumulation or greater protein stability.

Total RNA treated with the DNase extracted from mature leaves of two plants expressing the highest recombinant protein levels for each construct has been retro-transcribed and subjected to a real time RT-PCR analysis using primer designed to 3′ of GAD65mut (GADh1: GTTTGGAGTTGGCAGAGTAAT (SEQ ID NO: 16), GADh2: AGACATTTGTGTGCTGAGG) (SEQ ID NO: 17) sequences.

cDNA amounts have been calculated using the Gene Amp 5700 Sequence Detector (Perkin Elmer) kit. All the quantifications have been normalized to actin cDNA fragments using ACT1: ATCCCAGTTGCTGACAATAC (SEQ ID NO: 18) and ACT2: GGCCCGCCATACTGGTGTGAT (SEQ ID NO: 19) primers.

### Results

In the following tables (from 2 to 4) and figures (from 5 to 7) protein expression data of samples from all the analyzed constructs are reported; GAD percentage has been calculated with respect to extracted soluble proteins.

Particularly FIG. 2 shows GAD65 expression levels in plants transformed with G65mut construct.

TABLE 2
GAD65mut expression data

| | |
|---|---|
| Expression average value: | 0.25 |
| Expression highest value: | 2.2 |
| Minimal value: | 0.006 |
| Standard deviation: | 0.40 |

FIG. 3 shows GAD65 expression levels in plants transformed with G67/65mut construct (48 total plants transformed). Particularly:

TABLE 3
GAD67/65mut expression data

| | |
|---|---|
| Expression average value: | 0.26 |
| Expression highest value: | 2.4 |
| Minimal value: | 0.007 |
| Standard deviation: | 0.40 |

FIG. 4 shows GAD65 expression levels in plants transformed with G65mut (37 total plants transformed). Particularly:

TABLE 4
GAD65 expression data

| | |
|---|---|
| Expression average value: | 0.096 |
| Expression highest value: | 0.28 |
| Minimal value: | 0.001 |
| Standard deviation: | 0.072 |

FIG. 5 shows a comparison of GAD65mut expression levels, as percentage of total soluble proteins, for the plants expressing the highest levels of recombinant protein for each used construct. Expression levels of recombinant protein have been estimated by RIA analysis.

The system described for GAD in planta expression allowed remarkably higher expression levels of mutated recombinant protein than those obtained using not mutated GAD65 to be obtained.

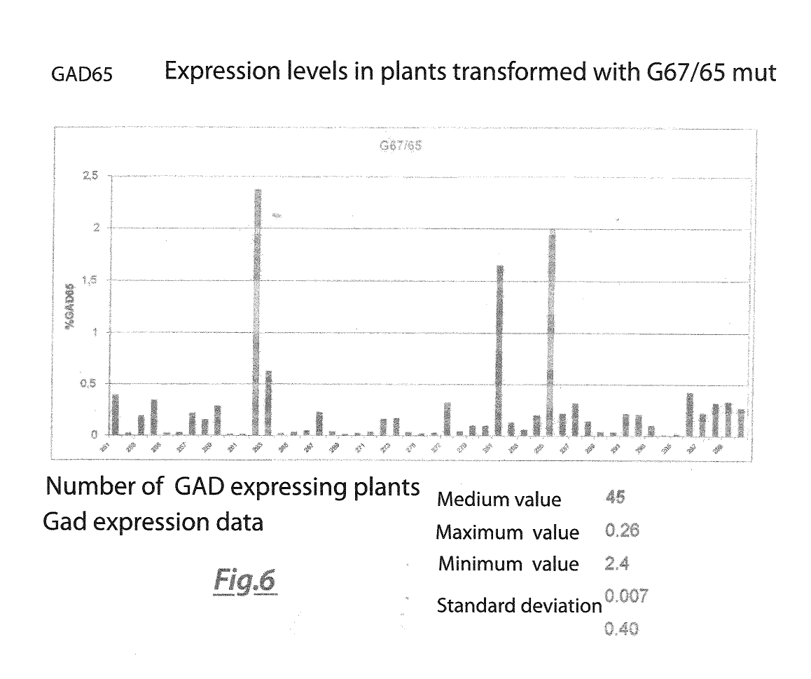

In FIG. 6 results of real time RT-PCR analysis are reported. Particularly, the Figure displays the quantification of GAD65mut transcript relating to actin in plants expressing the highest levels of recombinant GAD65mut, transformed with GAD65mut (204, 206), GAD67/65mut (262, 285) and GAD65 (331, 332). The results of this analysis demonstrate that there are no differences of the transcript relative amounts in analyzed transgenic plants.

Therefore it is possible to conclude that the differences in the observed protein expression levels do not result from differences of transcript accumulation levels. The difference of recombinant protein accumulation levels therefore can result from greater stability of the recombinant protein.

### EXAMPLE 3

### GAD65mut Purification in Plant

### Materials and Methods

### Purification Procedure

From the plant expressing the recombinant protein highest levels a strategy for the purification of recombinant protein has been provided. Plant leaves from self-fertilization off-spring of GAD65mut expressing 206 plant have been used in these experiments.

Total soluble proteins have been extracted using an extraction buffer consisting of: phosphate 50 mM pH 8.0 and Tween20 0.5%. Leaf tissue has been ground with pestle in liquid nitrogen and successively homogenised in buffer at buffer:leaf tissue 1:3 ratio. Then centrifugation has been carried out for 30° at 15000 rpm. The supernatant has been used in the following steps. Firstly the supernatant has been dialysed over night against sodium phosphate 25 mM pH 7.5 buffer and successively loaded on DEAE Sepharose column. Then the column has been eluted with the same buffer added with 0.1, 0.2 and 0.4 M NaCl. Different obtained fractions have been loaded on gel and the gel has been stained with Coomassie Blue and successively used for Western blot analysis; the analysis result are reported in FIG. 11.

Fraction eluted using NaCl 0.4 M added buffer has been dialysed using Hepes pH 7.9 buffer. This dialysed fraction successively has been loaded on gel filtration S200 and S75 columns for further purification.

### BIBLIOGRAPHY

Hagopian W A, Michelsen B, Karlsen A E, Larsen F, Moody A, Grubin C E, Rowe R, Petersen J, McEvoy R, Lernmark A. Diabetes. 1993 42(4):631-6.
Baekkeskov S, Nielsen J H, Marner B, Bilde T, Ludvigsson J, Lernmark A. Nature. 1982, 298(5870):167-9.
Porceddu, A. Falorni, N. Ferradini, A. Cosentino, F. Calcinaro, C. Faleri, M. Cresti, F. Lorenzetti, P. Brunetti and M. Pezzotti. Molecular Breeding. 1999, 5: 553-560.
Avesani L, Falorni A, Tornielli G B, Marusic C, Porceddu A, Polyerari A, Faleri C, Calcinaro F, Pezzotti M. Transgenic Research. 2003, 12:203-212.
Hampe C S, Hammerle L P, Falorni A, Robertson J, Lernmark. FEBS Lett. 2001 488(3):185-9.
Borgese N, Gazzoni I, Barberi M, Colombo S, Pedrazzini E. Mol Biol Cell. 2001 August; 12(8):2482-96.

1. Method for the production in plant of a mutated form of glutamic acid decarboxylase autoantigen (GAD65mut) or a chimera thereof, comprising the following steps:
a) transformation of a plant or a portion or tissue thereof, with an expression vector comprising the sequence of human GAD65 mutated form, characterised by the substitution of Lys aminoacid at position 396 with Arg aminoacid, under the control of a plant constitutive promoter and terminator using Agrobacterium tumefaciens;
b) plant growth and auto-antigen expression;
c) auto-antigen expressing plant tissue harvesting.

2. Method according to claim 1, further comprising a purification step by column chromatography of the autoantigen (GAD65) mutated form, from step c) harvested plant tissue.

3. Method according to claim 1, wherein said chimera is GAD67/65mut (SEQ ID NO:6).

4. Method according to claim 1, wherein said plant is tobacco plant.

5. Method according to claim 1, wherein said plant tissue is leaf.

6. Method according to claim 1, wherein said promoter is CaMV P35S and said terminator is CaMV T35S.

7. Use of plant or plant tissue transformed using the expression vector comprising the sequence of human GAD65 mutated form or chimera thereof, characterised by the substitution of Lys aminoacid at position 396 with Arg aminoacid, under the control of a plant constitutive promoter as a bioreactor for the production of GAD65 autoantigen;

8. Use according to claim 7, wherein said chimera is GAD67/65mut (SEQ ID NO:6).

9. Use according to claim 7, wherein said plant is tobacco plant.

10. Use according to claim 7, wherein said plant tissue is leaf.

11. Expression vector comprising the sequence of human GAD65 mutated form or a chimera thereof, characterised by the substitution of Lys aminoacid at position 396 with Arg aminoacid, under the control of a plant constitutive promoter and terminator.

12. Expression vector according to claim 11, wherein said chimera is GAD67/65mut (SEQ ID NO:6).

13. Expression vector according to claim 11, wherein said constitutive promoter is CaMV P35S CaMV and said terminator is CaMV T35S.

14. Prokaryotic or eukaryotic competent cells transformed using the expression vector according to claim 11.

15. Competent cells according to claim 14, wherein said prokaryotic cells are from Agrobacterium tumefaciens or Escherichia colit.

16. Use of the expression vector according to claim 11, for the transformation of a plant or plant portion.

17. Use according to claim 16, wherein said plant is tobacco plant.

18. Use according to claim 16, wherein said plant tissue is leaf.

19. A competent cell according to claim 14, for the transformation of a plant or plant portion.
